(19) United States
(12) Patent Application Publication
(10) Pub. No.: US 2005/0089472 A1
(43) Pub. Date: Apr. 28, 2005

(54) Monitoring the status of a condition of an animal through visually observable indicia

(76) Inventors: Aaron H. Shovers, Long Beach, CA (US); Steven C. Sereboff, Westlake Village, CA (US)

Correspondence Address: SOCAL IP LAW GROUP, 310 N. WESTLAKE BLVD. STE 120, WESTLAKE VILLAGE, CA, 91362, US

(21) Appl. No.: 10/692,364

(22) Filed: Oct. 22, 2003

(51) Int. Cl. A61K 49/00

(52) U.S. Cl. 424/9.1

(57) ABSTRACT

There are disclosed methods and apparatus for monitoring the status of a condition of an animal. A condition is selected for monitoring. A monitoring material is selected for introduction into the animal. A method of introducing the monitoring material into the animal is selected. At least one site on the animal is selected for introducing the monitoring material. The monitoring material is introduced into the animal at the selected sites. As a result of the introduction of the monitoring material, a visually ascertainable indication of a status of a condition of the animal appears on the animal's skin. The indication may be observed to thereby visually ascertain the status of the condition.

### RELATED APPLICATION INFORMATION

This application claims the benefit of Provisional Application No. 60/449,308 Feb. 21, 2003, which is incorporated herein by reference.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by any one of the patent disclosure as it appears in the Patent and Trademark Office patent files or records, but otherwise reserves all copyright and trade dress rights whatsoever.

### BACKGROUND OF THE INVENTION

1. Field of the Invention

The present invention relates to monitoring the status of a condition of an animal.

2. Description of Related Art

The human body has immunity. Immunity is “the condition of being protected against any diseases or poisons.” Immunity is the power which an individual acquires to resist or overcome an infection to which most of its species are susceptible. Immunity is the ability of an organism to resist disease.

The human body has resistance. Resistance is the more or less complete insusceptibility of an individual to various noxious agents such as poisons, toxins, irritants, bacteria, viruses, fungi, allergens, proteins, protein fragments, etc.

Attenuated means “a deliberate thinning or weakening of toxicity of a virus or micro-organism, by using heat, chemicals, repeated inoculation, or successive culturing.”

There are certain body tissues that are designed to combat diseases and deal with allergies. When first invaded by a foreign substance (called allergens) or organisms (such as viruses, bacteria, or fungi), the body is alerted to the imminent danger by the release of histamines and other substances. This brings forth the defense mechanisms of the body, by attracting phagocytes to the invaded area. (Phagocytes are specialized white blood cells which physically battle and consume the invading viruses, bacteria, or fungi.) If an allergic person is affected by a specific thing to which he is allergic (i.e., an allergen), antibodies (secretions from lymphocytes) attach themselves to that specific allergen. In both of these cases, the result would be a reaction called “inflammation”. Inflammation typically manifests itself by increased local blood flow, capillary fragility, redness, swelling, fever, pain, or even the rupture of cells, with bleeding, at the site being invaded. Examples of such inflammation include herpes sores and blood-shot eyes.

Whereupon, with adequate immunity, the attacked host would be strong enough to ward off these harmful things or neutralize them. Or if the attacked host has a low immunity, the offending agents could go on to do great damage, or even cause the death of that person.

It is believed that sixty-five percent of all people are unaware of what is going on in their bodies. By the time they realize they have a problem, the problem has advanced into a disaster, or the person has died. Every healthy human is living with many virulent bacteria, funguses, and viruses which can only attack tissues after they have been weakened by systemic illnesses, genetic defects, poor diet, mental stress or physical abuse.

### DESCRIPTION OF THE DRAWINGS

FIG. 1 is a flow chart of a method of the invention.

FIGS. 2A-2E show a series of monitoring marks in accordance with the invention.

FIG. 3 is a graph of appearance levels for monitoring marks in accordance with the invention.

FIG. 4 shows an antagonist or agonist and a tag in accordance with the invention.

FIG. 5 shows culture films in accordance with the invention.

FIG. 6 shows a cup in accordance with the invention.

FIG. 7 shows a semipermeable membrane in accordance with the invention.

FIG. 8 shows a tooth in accordance with the invention.

FIG. 9 shows a nail bed in accordance with the invention.

### DETAILED DESCRIPTION OF THE INVENTION

Throughout this description, the embodiments and examples shown should be considered as exemplars, rather than limitations on the apparatus and methods of the present invention.

The invention provides a number of benefits. Simply through observation, it is easy to detect the various effects certain organisms, foods, vitamins, medicines, radiation, chemicals, exercise, mental states, lack of sleep or rest had upon a person's immunity. The invention enables a person to take immediate steps to avoid oncoming diseases, before they become disastrous.

The invention is applicable to many kinds of animals, including humans. It may be used on most mammals, birds and amphibians. Because of their thicker skins or scales, it may be more difficult to monitor the condition of reptiles and fish in accordance with the invention. Thus, for some animals it may be necessary to remove such matter as scales or feathers.

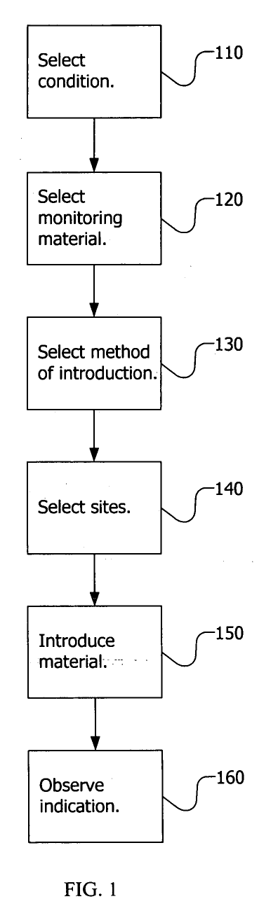

Referring now to FIG. 1, there is shown a flow chart of a method of the invention. As an initial matter; a condition is selected for monitoring (step 110). This condition may be immunity, ability to produce antibodies, general or specific disorders of the immune system, or the general condition of the animal. Certain types of arthritis, disorders of the immune system, and autoimmune diseases (like Lupus) could also be monitored to learn the status of disease, determine which medication is best, and what dose of medication works most effectively.

Even small changes in an animal's condition may be especially important in some situations. For example, immunity and other conditions must be carefully observed after organ transplantation, implantations, open heart surgery, or when the skin is severely burnt. The main problem with tissue and organ transplantation or implantation is rejection of the transplant, as matter foreign to the host's body has been introduced. For example, to successfully transplant a tissue, an organ, or blood from one person to another, it would be helpful if the effects of anti-rejection medications could be constantly monitored in a simple straight-forward manner, to achieve the best choice of drugs and the best dosage to avoid overmedication of the person who has received a transplant.

Another application is to monitor the inability of an animal to produce antibodies (or other disorders of the immune system—like genetic disorders). With such monitoring, it would be simple to test different drugs, foods, vitamins, or minerals that increase (or diminish) an animal's ability to produce antibodies, or neutralize an offending agent.

The method of the invention may be used for monitoring many other conditions. The conditions include hormone levels, temperature and blood pressure. The conditions may also be used to detect exposure to radiation, temperature, lack of sleep, mental stress, or other externalities. Furthermore, the condition may be presence or levels of certain proteins, amino acids, microbes, toxins, drugs, minerals (e.g., iron, calcium, magnesium, etc.), vitamins (e.g., A, B, C, E), insulin, hormones (e.g., estrogen, testosterone, thyroid, growth, adrenal), fat tissue, cholesterol, triglycerides, and antibodies.

In one step, a monitoring material is selected for introduction into the animal (step 120). The monitoring material may be, for example, certain chemicals, a naturally weak organism or an attenuated virus, fungus or bacteria. Other organic and inorganic irritants may be used, depending on the animal and selected condition. Both organic and inorganic allergens may be used as a monitoring material.

In another step, a method of introducing the monitoring material into the animal is selected (step 130). The method of introduction may be, for example, injecting the monitoring material just under the skin, such as through inoculation.

Alternatively, the monitoring material may be introduced through absorption by the skin. For example, the monitoring material or a precursor may be comprised in a lotion or cream. The lotion or cream may be applied to the animal's skin at the selected sites. The monitoring material may be applied to or into the skin through stamping or tattooing techniques. Furthermore, the monitoring material may be applied in conjunction with a tattoo. In one embodiment, an ordinary tattoo is applied to a conspicuous site, and the monitoring material implanted so that as the quality to be monitoring changes, the appearance at the tattoo site changes (e.g., a tattoo of an eye appears to close, or a tattoo of a thermometer appears to rise).

Other methods of introduction may be used. Where the introduction site is a tooth, the monitoring material may be applied on or in the tooth, for example with a filling, veneer, bond or crown. The monitoring material may also be introduced through ingestion or transplantation.

For some monitoring materials, it may be desirable to take steps to keep the monitoring material localized or in place, or to at least partially insulate the monitoring material from surrounding tissue. For example, it may be desirable to circumscribe the monitoring material within a cellular substrate or inert material such as metal, ceramic, composite or plastic, or it may be introduced in a place or in a way that prevents or moderates dissipation. A culture plate, tissue sandwich, osmotic membrane or semi-permeable membrane may also be used. A combination of techniques and materials may be used to insulate the monitoring material from surrounding tissue. On the other hand, it may be desirable for the monitoring material to dissipate after a period or change of condition.

It may be desirable to select more than one monitoring material. For example, four different monitoring materials may be introduced, each one producing a different visual indicator with a change in the selected condition.

In another step, at least one site on the animal for introducing the monitoring material is selected (step 140). Depending on the animal and the quality of its skin, it may be desirable to select sites which are relatively hairless, relatively free of blemish, or otherwise clear. It may also be desirable to select a site which is self-limiting for the monitoring material. It may further be desirable to select sites which are either conspicuous or inconspicuous. For example, for a human, a site such as the back of the hand may be desirable for ease of observation, or a site such as the arm pit, the tongue or a tooth for privacy. Other tissues which might be suitable include teeth, finger or toe nails, or under a nail bed. The monitoring site may be tagged, for example with color or a radioative ion.

It should be recognized that these steps 120, 130, 140 may be in any order. Furthermore, the selection of one or two of these (i.e., monitoring material, method of introduction, and sites) may impact the selection of the others. In addition, there may be a step of preparing the sites for introducing the monitoring material. As suggested above, this may include tattooing the sites, or doing other things such as treating with medications or other matter, de-scaling, de-feathering, brushing, scraping, heating or cooling.

Next, the monitoring material is introduced into the animal at the selected sites (step 150). As a result of the introduction of the monitoring material, a visually ascertainable indication of a status of the selected condition will appear on the animal's skin. Accordingly, the indication may be observed to thereby visually ascertain the status of the selected condition (step 160). Visual ascertainment may be enhanced or made possible by shining light or other radiation (e.g., white light, ultraviolet light, infrared light, X-rays) on the monitoring site, or by applying a second material (e.g., a cream or lotion) which makes the condition apparent. Visual ascertainment may also be enhanced or made possible through use of optical filters (e.g., colored glasses).

The introduction may be made to provide a visual indicator with a definite shape, such as two dots, a triangle or a square. More complex patterns such as the points of a clock for different conditions, may also be used. Visual observation may be obtained from changes in color, shape, size, swelling, bleeding, pattern, texture. Furthermore, a combination of these qualities may be combined for monitoring a single condition, or even a number of conditions. The change in appearance may proceed either forward (as discussed above) or in reverse. In a forward paradigm, a normal appearance reflects a normal state of the condition, and a changed appearance reflects a changed state of the selected condition. In a reverse paradigm, an unusual appearance reflects a normal state of the condition and a normal appearance reflects a changed state of the condition. The visual indicator changes when the selected condition of the animal changes.

Monitoring may be performed or enhanced using contact or contactless devices having mechanical and/or electronic components (e.g., a computerized read out). Such a monitoring device may be used to detect changes with a finer sensitivity, or to detect changes which might not otherwise be visible.

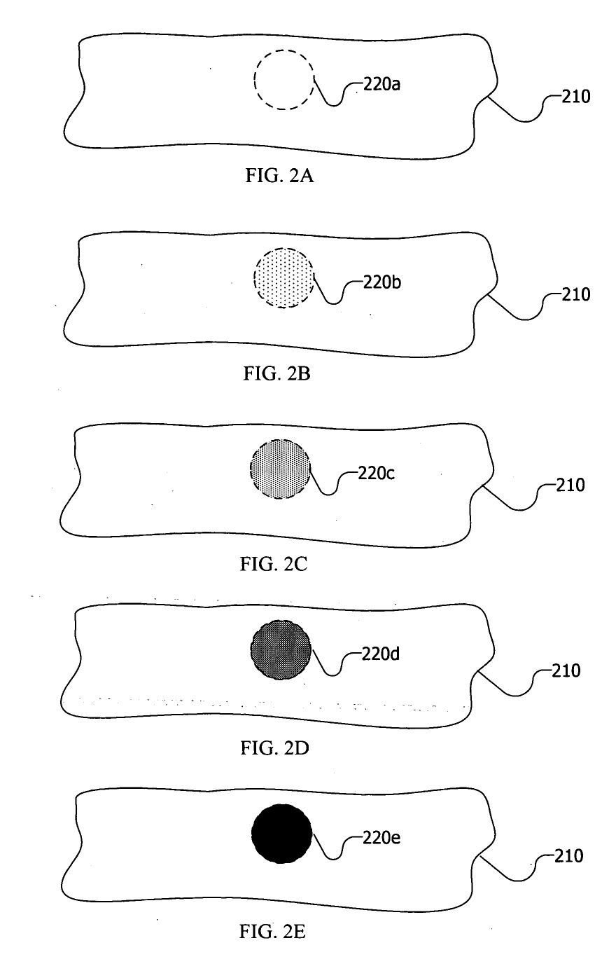

Referring now to FIGS. 2A-2E, there is shown a series of monitoring marks showing a progressive change in the monitored condition. As mentioned above, after selecting at least one site 210 on the animal, introducing a monitoring material into the animal at the selected sites, the selected sites may be periodically observed. As a result of the introduction of the monitoring material, a visually ascertainable indication of the selected condition of the animal appears on the animal's skin. In the embodiment of FIGS. 2A-2E, the condition trends from normal to poor, and the indicia trends from inconspicuous to very conspicuous.

If the monitored condition is normal, then there is no observable change in the site. In the case of immunity, normal may equate with strong (80-100%). Thus, the site 220a (FIG. 2A) may appear normal, or indistinguishable from adjacent tissue.

If the monitored condition varies a small amount from normal, then there may be a small observable change in the site. For example, if the site 220b appears swollen (FIG. 2B), then the animal's immunity may be weakening (60-80%).

If the monitored condition varies a small amount more from normal, then there may be a more observable change in the site. For example, if the site 220c appears light pink (FIG. 2C), then the animal's immunity may be weakening (40-60%).

If the monitored condition varies a larger amount from normal, then there may be a larger observable change in the site. For example, if the site 220d is red and inflamed (FIG. 2D), then the immunity state might be rated as weak (20-40%).

If the monitored condition varies an extreme amount from normal, then there may be a very easily observable change in the site. For example, if the animal's immunity is between 0% and 20%, then the selected site 220e may have open sores, outright bleeding or be black (FIG. 2E). Of course, if immunity drops too low, the animal may be suffering from severely poor health. In that case, the animal may even perish before the indication reaches that point. In this way, at least five different states (e.g., levels of a condition) may be observed.

By this means, an animal's condition may be visually ascertained. There may be a shape on or under the skin which changes depending on the immune level.

For many elements, chemicals, hormones, enzymes, proteins, bacteria, viruses, fungi and allergens, there are other elements, chemicals, hormones, enzymes, proteins, bacteria, viruses, fungi and allergens which can interact to support the ongoing existence of the first entity or slow down, neutralize or even stop the existence of the first entity. As used herein, an “antagonist” is anything (e.g., chemical, protein, bacterial, viral) which neutralizes, nullifies or refused to interact with some other things (e.g., another chemical, protein, bacterial, viral). An “agonist” is anything (e.g., chemical, protein, bacterial, viral) which energizes, intensifies or readily interacts with some other things (e.g., another chemical, protein, bacterial, viral). Thus, according to one aspect of the invention, there is a methodology for using an animal's own chemical makeup and defense mechanisms to constantly monitor various conditions of the animal's tissues. According to another aspect of the invention, interactions of various antagonists or agonists are monitored.

In one embodiment of the invention, diabetes is monitored. However, the invention is not limited to monitoring diabetes. The same methodology may be used to monitor other conditions for which an antagonist or agonist is found. These conditions may include overexertion, lack of sleep, depression, allergies, diseases, alcoholism and hormonal imbalances.

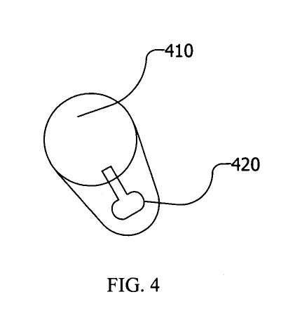

After selecting a specific condition to monitor, it is necessary to determine the specific causes and effects of that condition. In a diabetic patient, it is sugar levels and insulin levels that require monitoring. Secondly, it is necessary to discover an antagonist which strongly refuses to interact with or agonist which strongly interacts with one or more of the causal factors. After selecting an antagonist or agonist, the material is tagged with a color or irritant. Referring now to FIG. 4, there is shown an antagonist or agonist 410 and a tag 420. The tagged antagonist or agonist may then be localized. Localization may be within a tissue culture sandwich, a cup made from physiologically inert material, a permeable membrane, a semi-permeable membrane, a tooth restoration, a specific body tissue or combinations of these.

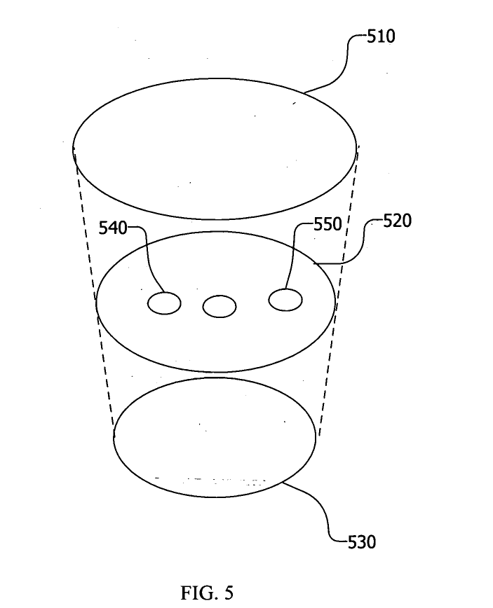

Referring now to FIG. 5, there are shown three culture films 510, 520, 530 with two conditions being monitored at sites 540, 550. For example, one site 540 might monitor sugar, and the other 550 insulin. The culture films 510, 520, 530 have a diameter of 4 mm. The sites 540, 550 have a diameter of 1 mm. The top culture film 510 may be spaced 12 mm from the bottom culture film 530.

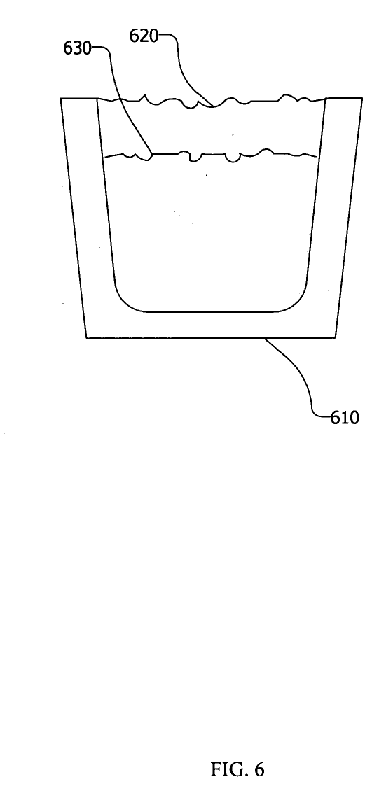

Referring now to FIG. 6, there is shown a cup 610 having a clear cover 620 over a monitoring material 630. The cup 610 may be 1½ mm in diameter at its widest point, and ½ mm tall.

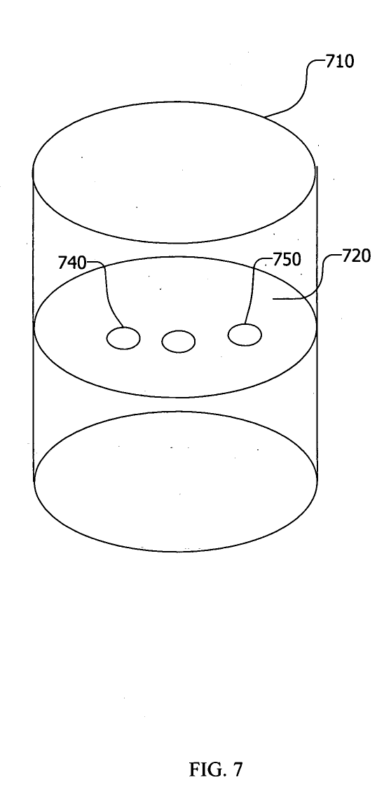

Referring now to FIG. 7, there is shown a semipermeable membrane 710 including a matrix 720 to hold monitoring material 740, 750.

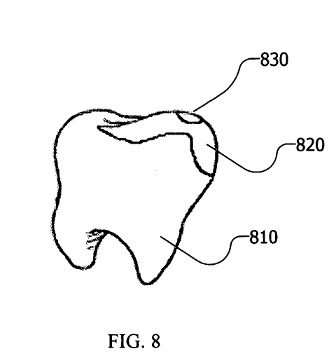

Referring now to FIG. 8, there is shown a tooth 810 having a tooth restoration 820 which includes a monitoring material 830 in a permeable material.

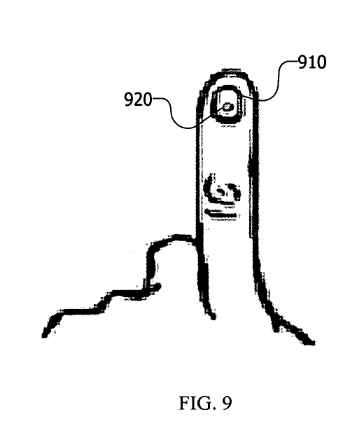

Referring now to FIG. 9, there is shown a nail bed 910 with a monitoring material 920.

As shown, the implant material or transplant tissue may be placed within the body of the animal. In the diabetes embodiment, after healing, the patient could eat a heavy meal or exercise while the patient's monitoring site is periodically watched. The patient may do this himself. If the patient's body is accumulating too much sugar, the sugar-monitoring spot would change from colorless, to weak color, and finally to dark color for example, showing weak to strong coloration. If the patient required insulin, the patient's insulin-monitoring spot would change from colorless, to weak color, and finally to strong dark for example.

In a similar fashion, other conditions may be monitored by observing the overlaying tissues changes from normal, flat tissues to raised tissues, to swollen tissues, to red inflamed tissues, to open sores, to necrotic tissue, as the monitored condition got worse. The size, swelling, inflammation and color changes of the monitored area may be monitored by using graphic charts or electronic devices.

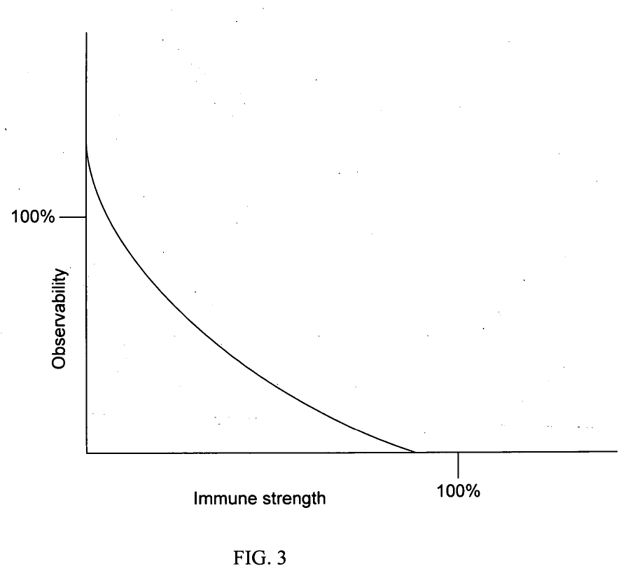

Referring now to FIG. 3, there is shown a graph of appearance levels for monitoring marks in accordance with the invention. The graph shows that for a typical monitoring material in a typical animal, it is expected that observability and immunity will show an inverse relationship. On the other hand, it is believed that with an appropriate combination of monitoring material, implantation method, animal and site, observability and immunity may show a direct relationship. It is expected, in addition, that a desired observation may be obtained for a given quality through appropriate selection of monitoring material, implantation method and site.

As can be seen, the invention provides methods for monitoring a condition of an animal. In one embodiment, the animal's immunity may be monitored. When implemented, it can be easily determined if a person is (or is not) able to produce adequate defense mechanisms to protect itself. By measuring the ongoing changes of an animal's immune system, certain precautions can be taken each time the animal's immune system becomes weakened from any cause. Thus, the animal's basic health could be maintained and it will be assured a healthier and longer life.

Although exemplary embodiments of the present invention have been shown and described, it will be apparent to those having ordinary skill in the art that a number of changes, modifications, or alterations to the invention as described herein may be made, none of which depart from the spirit of the present invention. All such changes, modifications and alterations should therefore be seen as within the scope of the present invention.

1. A method of monitoring a condition of an animal, the animal having a visually observable tissue, the method comprising
selecting a monitoring material for introduction into the animal
selecting a method of introducing the monitoring material into the animal
selecting at least one site on the animal for introducing the monitoring material
introducing the monitoring material into the animal at the selected sites, wherein as a result of the introduction of the monitoring material, a visually ascertainable indication of a status of the condition of the animal appears in at least one pre-defined site on the animal
observing the indication to thereby visually ascertain the status of the condition.

2. The method of monitoring a condition of an animal of claim 1 further comprising selecting a method of localizing the monitoring material.

3. The method of monitoring a condition of an animal of claim 1, wherein animal has a nail tissue, and the site is under the nail tissue.

4. The method of monitoring a condition of an animal of claim 1, wherein animal has a mouth, and the site is within the mouth.

5. The method of monitoring a condition of an animal of claim 4, wherein the mouth includes at least one tooth, and the site is on the tooth.

6. The method of monitoring a condition of an animal of claim 4, wherein the mouth includes a cheek, and the site is the cheek.

7. The method of monitoring a condition of an animal of claim 1 further comprising selecting a condition to monitor.

8. The method of monitoring a condition of an animal of claim 1 wherein the site is easily monitored.

9. The method of monitoring a condition of an animal of claim 1 wherein the monitoring material is selected from the group consisting of: organic materials and inorganic materials.

10. The method of monitoring a condition of an animal of claim 1 wherein the method of implanting is selected from the group consisting of transplantation, implantation, injection, tattooing and absorption.

11. The method of monitoring a condition of an animal of claim 1 wherein the monitoring material is comprised in a lotion, and the step of introducing the monitoring material comprises applying the lotion to the animal's skin.

12. The method of monitoring a condition of an animal of claim 1 wherein the step of selecting at least one site comprises selecting sites which are relatively hairless.

13. The method of monitoring a condition of an animal of claim 1 wherein the step of selecting at least one site comprises selecting sites which are relatively free of blemish.

14. The method of monitoring a condition of an animal of claim 1 further comprising preparing the sites for introducing the monitoring material.

15. The method of monitoring a condition of an animal of claim 1 wherein the steps of (a) selecting a monitoring material, (b) selecting a method of introducing and (c) selecting at least one site are performed in the order (b), (c), (a).

16. A method of monitoring a condition of an animal, the animal having a visually observable tissue, the method comprising
selecting a monitoring material for introduction into the animal
selecting a method of introducing the monitoring material into the animal
selecting at least one site on the animal for introducing the monitoring material
introducing the monitoring material into the animal at the selected sites, wherein as a result of the introduction of the monitoring material, a visually ascertainable indication of a status of the condition appearing on the tissue.

17. The method of monitoring a condition of an animal of claim 16 further comprising selecting a condition to monitor.

18. The method of monitoring a condition of an animal of claim 16 wherein the monitoring material is selected from the group consisting of: living tissues, organic materials and inorganic materials.

19. The method of monitoring a condition of an animal of claim 16 wherein the method of introducing is selected from the group consisting of injection, tattooing and absorption.

20. The method of monitoring a condition of an animal of claim 16 wherein the monitoring material is comprised in a lotion, and the step of introducing the monitoring material comprises applying the lotion to the animal's skin.

21. The method of monitoring a condition of an animal of claim 16 wherein the step of selecting at least one site comprises selecting sites which are relatively hairless.

22. The method of monitoring a condition of an animal of claim 16 wherein the step of selecting at least one site comprises selecting sites which are relatively free of blemish.

23. The method of monitoring a condition of an animal of claim 16 further comprising preparing the sites for introducing the monitoring material.

24. A method of monitoring a condition of an animal, the animal having a visually observable tissue, the method comprising
selecting at least one site on the animal
introducing a monitoring material into the animal at the selected sites
periodically observing the selected sites, wherein as a result of the implantation of the monitoring material, a visually ascertainable indication of a status of the condition appears on the animal's skin
wherein if the status of the condition is normal, then the selected site has a first appearance
wherein if the status of the condition is changed a small amount from normal, then the selected site has a second appearance which appears to be a small change from the first appearance
wherein if the status of the condition changes considerably from normal, then the selected site has a third appearance which appears very noticeably different from the first appearance.

25. The method of claim 18 wherein if the status of the condition changes a dangerous amount from normal, then the selected site has a fourth appearance which is especially conspicuous.

26. The method of claim 18 further comprising selecting the condition to monitor.
